(19) United States
(12) Patent Application Publication
(10) Pub. No.: US 2024/0317793 A1
(43) Pub. Date: Sep. 26, 2024

(54) NUCLEOSIDE TRIPHOSPHATES WITH MODIFIED PHOSPHATE CHAINS, AND METHODS OF SYNTHESIZING THE SAME

(71) Applicant: Illumina, Inc., San Diego, CA (US)

(72) Inventors: Hassan Bohra, Singapore (SG); Xiangyuan Yang, Singapore (SG); Ramesh Neelakandan, Singapore (SG); Yin Nah Teo, Singapore (SG); Abdul Sadeer Abd Salam, Singapore (SG); Eric Murtfeldt, San Diego, CA (US)

(73) Assignee: Illumina, Inc., San Diego, CA (US)

(21) Appl. No.: 18/608,556

(22) Filed: Mar. 18, 2024

Related U.S. Application Data
(60) Provisional application No. 63/491,125, filed on Mar. 20, 2023.

(51) Int. Cl. C07H 21/00

(52) U.S. Cl. CPC C07H 21/00

(57) ABSTRACT

In some examples, a nucleoside triphosphate analogue may include a sugar, a nucleobase coupled to the sugar, a triphosphate group coupled to the sugar, a heteroatom coupled to an alpha phosphate of the triphosphate group, and a first substituent coupled to the heteroatom. The heteroatom may be selected from the group consisting of oxygen, nitrogen, and carbon. The first substituent may include at least one of an alkyl chain or a polymer.

### CROSS-REFERENCE TO RELATED APPLICATIONS

This application claims the benefit of U.S. Provisional Patent Application No. 63/491,125, filed Mar. 20, 2023 and entitled “Nucleoside Triphosphates with Modified Phosphate Chains, and Methods of Synthesizing the Same,” the entire contents of which are incorporated by reference herein.

### FIELD

This application relates to nucleoside triphosphates.

### BACKGROUND

Modified nucleoside triphosphates are key drivers of various sequencing technologies. For example, some sequencing technologies track the incorporation of fluorophore-labelled nucleoside triphosphates. The sequence of DNA is determined by reading the emission from nucleotide-specific fluorophores during each incorporation cycle.

It may be desirable to modify nucleoside triphosphates to include different moieties. Traditional methods of installing modifications may use intermediates that are costly and involve multi-step syntheses. Moreover, extensive functionalization of triphosphate substrates carries a risk of phosphate chain degradation. Such degradation can lead to poor overall yields.

### SUMMARY

Nucleoside triphosphate analogues, and methods of making the same, are provided herein.

Some examples herein provide a nucleoside triphosphate analogue. The nucleoside triphosphate analogue may include a sugar; a nucleobase coupled to the sugar; a triphosphate group coupled to the sugar; a heteroatom coupled to an alpha phosphate of the triphosphate group; and a first substituent coupled to the heteroatom.

In some examples, the heteroatom may be selected from the group consisting of oxygen, nitrogen, and carbon.

In some examples, the first substituent may include at least one of a fluorescent dye, an alkyl chain, and a polymer. In some examples, the polymer may include at least one of a synthetic peptide and a synthetic polymer. The synthetic polymer may include an ethylene glycol polymer. In some examples, the first substituent may include about 1 to about 50 repeating units.

In some examples, the first substituent may further include a functional group. In some examples, the functional group may include at least one of an amine, an azide, a carboxylic acid, a thiol, a tetrazine, a cyclooctyne, and an alkyne. In some examples, the sugar may include a natural ribose or deoxyribose.

In some examples, the nucleoside triphosphate analogue may further include a second substituent coupled to the sugar via a 3′ carbon of the sugar. In some examples, the second substituent may include a protecting group. In some examples, the protecting group may include at least one of a vinyl group, an allyloxy methyl group, and an azido methyl group.

In some examples, the nucleoside triphosphate analogue may further include a third substituent coupled to the sugar via a 2′ carbon of the sugar. In some examples, the third substituent may include at least one of a fluorine, a methoxy group, and an ethoxy group.

In some examples, the nucleoside triphosphate analogue may further include a fourth substituent coupled between a beta phosphate and a gamma phosphate of the triphosphate group. In some examples, the fourth substituent may include at least one of an oxygen atom, a nitrogen atom, a methylene, a fluoromethylene, a difluoromethylene, a sulfur atom, and a selenium atom.

In some examples, the nucleobase be a non-naturally occurring nucleobase.

Some examples herein provide a method of synthesizing a nucleoside triphosphate analogue. In some examples, the method may include coupling a phosphorylating agent to a sugar of a nucleoside. The nucleoside may include the sugar and a nucleobase. The phosphorylating agent may include a protecting group and a substituent. In some examples, the method may include oxidizing the coupled phosphorylating agent; and removing the protecting group from the oxidized, coupled phosphorylating agent to form a monophosphate diester that includes the substituent. The method further may include activating the monophosphate diester; and reacting the activated monophosphate diester with a pyrophosphate to form a triphosphate group that includes the substituent.

In some examples, the sugar may include a protected 3′-O group.

In some examples, the substituent is coupled to an alpha phosphate of the triphosphate group. Illustratively, the substituent may be coupled to an alpha phosphate of the triphosphate group. In one nonlimiting example, the heteroatom is oxygen.

In some examples, the substituent may include at least one of a fluorescent dye, an alkyl chain, and a polymer. In some examples, the polymer may include at least one of a synthetic peptide and a synthetic polymer. In some examples, the synthetic polymer may include an ethylene glycol polymer. In some examples, the polymer may include about 1 to about 50 repeating units.

In some examples, the protecting group may include propionitrile.

In some examples, the phosphorylating agent may include a phosphoramidite. In some examples, the phosphorylating agent may be activated using 5-(ethylthio)-1H-tetrazole (ETT) and 4,5-dicyanoimidazole (DCI), tetrazole, or benzothiotetrazole. In some examples, the coupled phosphorylating agent may be oxidized using elemental iodine (I₂), t-butyl hydrogen peroxide (t-BuOOH), (1S)-(+)-(10-camphorsulfonyl)-oxaziridine (CSO), hydrogen peroxide, or meta-chloroperoxybenzoic acid (m-CPBA).

In some examples, the protecting group may be removed using 1,8-diazabicyclo[5.4.0]undec-7-ene (DBU). In some examples, the monophosphate diester may be activated using trifluoroacetic anhydride (TFAA) and N-methylimidazole (NMI).

Some examples herein provide another method of synthesizing a nucleoside triphosphate analogue. In some examples, the method may include activating a monophosphate group of a nucleotide. In some examples, the nucleotide may include a sugar, a nucleobase, and the monophosphate group. In some examples, the method may include reacting the activated monophosphate group with an amidation agent that includes a substituent, in an amidation reaction, to form an amidated monophosphate group that includes the substituent. The method may include activating the amidated monophosphate group. The method may include reacting the activated, amidated monophosphate group with a pyrophosphate to form a triphosphate group that includes the substituent.

In some examples, the sugar may include a protected 3′-O group.

In some examples, the substituent may be coupled to an alpha phosphate of the triphosphate group. In some examples, the substituent may be coupled to the alpha phosphate of the triphosphate group via a heteroatom. In some examples, the heteroatom is nitrogen.

In some examples, the substituent may include at least one of a fluorescent dye, an alkyl chain, and a polymer. In some examples, the polymer may include at least one of a synthetic peptide and a synthetic polymer. In some examples, the synthetic polymer may include an ethylene glycol polymer. In some examples, the polymer may include about 1 to about 50 repeating units.

In some examples, the amidation agent may include an amine. In some examples, the amidated monophosphate group may be activated using pyridine. In some examples, the amidated monophosphate group may be activated using carbonyldiimidazole (CDI).

Some examples herein provide another method of synthesizing a nucleoside triphosphate analogue. In some examples, the method may include activating a phosphorylating agent that includes a substituent. The method may include coupling the activated phosphorylating agent to a sugar of a nucleoside to form a monophosphate diester that includes the substituent. The method may include activating the monophosphate diester; and reacting the activated monophosphate diester with a pyrophosphate to form a triphosphate group that includes the substituents. In some examples, the nucleoside may include a sugar and a nucleobase.

In some examples, the sugar may include a protected 3′-O group.

In some examples, the substituent may be coupled to an alpha phosphate of the triphosphate group. In some examples, the substituent may be directly coupled to the alpha phosphate of the triphosphate group. In some examples, the substituent may include at least one of a fluorescent dye, an alkyl chain, and a polymer. In some examples, the polymer may include at least one of a synthetic peptide and a synthetic polymer. In some examples, the synthetic polymer may include an ethylene glycol polymer. In some examples, the polymer may include about 1 to about 50 repeating units.

In some examples, the phosphorylating agent comprises a phosphonate. In some examples, the phosphorylating agent is activated using N,N′-diisopropylcarbodiimide (DIC), N,N′-diisopropylethylamine (DIPEA), and 4-dimethylaminopyridine (DMAP). In some examples, the monophosphate diester is activated using carbonyldiimidazole (CDI) and pyridine.

Some examples herein provide another method of synthesizing a nucleoside triphosphate analogue. In some examples, the method may include reacting a monophosphate group of a nucleotide analogue with a morpholidation agent to form a phosphormorpholidate group that includes a first substituent. The nucleotide analogue may include a sugar, a nucleobase, and the monophosphate group including the first substituent. The method may include reacting the phosphormorpholidate with a modified with a modified bisphosphonic acid salt to form a triphosphate group including the first substituent. The modified bisphosphonic acid salt optionally may include a second substituent, in which case the triphosphate group also may include the second substituent.

In some examples, the first substituent is coupled to an alpha phosphate of the triphosphate group. In some examples, the first substituent is coupled to the alpha phosphate of the triphosphate group via a heteroatom. In some examples, the heteroatom is selected from the group consisting of oxygen, nitrogen, and carbon. In other examples, the first substituent is directly coupled to a phosphate atom of the alpha phosphate.

In some examples, the sugar may include a protecting group coupled to a 3′ carbon of the sugar.

In some examples, the first substituent may include at least one of a fluorescent dye, an alkyl chain, and a polymer. The polymer may include at least one of a synthetic peptide and a synthetic polymer. The synthetic polymer may include an ethylene glycol polymer. In some examples, the polymer may include about 1 to about 50 repeating units.

In some examples, the morpholidation agent may include morpholine. In some examples, reacting the monophosphate group with the morpholidation agent includes activating the monophosphate group with a dehydrating agent.

In some examples, the second substituent is coupled between beta and gamma phosphates of the triphosphate group. In some examples, the second substituent may be coupled to each phosphate group of the modified bisphosphonic acid salt. The second substituent may include an oxygen atom, a nitrogen atom, a methylene, a fluoromethylene, a difluoromethylene, a sulfur atom, or a selenium atom. In some examples, the modified bisphosphonic acid salt may include a tributylammonium counterion.

Some examples provided herein provide another method of synthesizing a nucleoside triphosphate analogue. In some examples, the method may include reacting a sugar of a protected nucleoside with a phosphite and a pyrophosphate to form a first intermediate. The protected nucleoside may include a sugar, a nucleobase, and a protected 3′-O group. The method may include reducing the first intermediate using a borane to form a boranophosphate intermediate. The method may include protecting the boranophosphate intermediate to form a protected boranophosphate intermediate. The method may include activating and reacting the protected boranophosphate intermediate with a reagent including a substituent to form a second intermediate that includes a protected triphosphate group that includes the substituent. The method may include deprotecting the second intermediate to form a triphosphate group that includes the substituent.

In some examples, the protected 3′-O group may include tert-butyldiphenylsilyl (TBDPS).

In some examples, the phosphite includes salicyl chlorophosphite. In some examples, the borane includes borane dimethylsulfide. In some examples, the boranophosphate intermediate is protected using a trimethylsilyl group.

In some examples, the substituent may include at least one of a fluorescent dye, an alkyl chain, and a polymer. In some examples, the polymer may include at least one of a synthetic peptide and a synthetic polymer. In some examples, the polymer may include an ethylene glycol polymer. In some examples, the polymer may include about 1 to about 50 repeating units.

In some examples, the reagent may include an alcohol group or an amine group.

It is to be understood that any respective features/examples of each of the aspects of the disclosure as described herein can be implemented together in any appropriate combination, and that any features/examples from any one or more of these aspects can be implemented together with any of the features of the other aspect(s) as described herein in any appropriate combination to achieve the benefits as described herein.

### BRIEF DESCRIPTION OF DRAWINGS

FIG. 1 illustrates nonlimiting examples of the present nucleoside triphosphate analogues.

FIG. 2 illustrates example nucleobases and example 3′-modifications that may be used in the present nucleoside triphosphate analogues.

FIGS. 3A-3C illustrate example modified nucleobases that may be used in the nucleoside triphosphate analogues.

FIG. 4 illustrates an example method of synthesizing a nucleoside triphosphate analogue.

FIG. 5 illustrates another example method of synthesizing a nucleoside triphosphate analogue.

FIG. 6 illustrates another example method of synthesizing a nucleoside triphosphate analogue.

FIG. 7 illustrates another example method of synthesizing a nucleoside triphosphate analogue.

FIG. 8 illustrates an example method of synthesizing a nucleoside triphosphate analogue through the use of boranophosphate intermediate.

FIG. 9 illustrates a flow of operations in an example method of synthesizing a nucleoside triphosphate analogue.

FIG. 10 illustrates a flow chart of operations in another example method of synthesizing a nucleoside triphosphate analogue.

FIG. 11 illustrates a flow chart of operations in another example method of synthesizing a nucleoside triphosphate analogue.

FIG. 12 illustrates a flow chart of operations in another example method of synthesizing a nucleoside triphosphate analogue.

FIG. 13 illustrates a flow chart of operations in another example method of synthesizing a nucleoside triphosphate analogue.

### DETAILED DESCRIPTION

Disclosed herein are nucleoside triphosphates with modified phosphate chains, and methods of synthesizing the same. It may be desirable to incorporate modifications into the phosphate chains of nucleoside triphosphates. For example, modifying the phosphate chains of nucleoside triphosphates may allow the tracking of the enzymatic synthesis of DNA. Illustratively, phosphate chains including fluorophores may be used to track the synthesis optically. Other modifications may allow other methods of tracking the synthesis.

As noted above, some previous methods for synthesizing modified nucleoside triphosphates use advanced intermediates that are costly and involve multi-step syntheses. Other previous methods involve the functionalization of triphosphate substrates. This can lead to phosphate chain degradation, which in turn may lead to poor overall yields. In comparison, the presently disclosed methods for synthesizing nucleoside triphosphates with a modified polyphosphate chain, and nucleotide triphosphates made thereby, do not require advanced intermediates. Instead, commercially available nucleosides or nucleotide monophosphates can be used as a starting point. Further, modifications that previously used multi-step syntheses can be prepared separately and installed en masse on the nucleoside using the methods provided herein. Still further, the number of steps after formation of the triphosphate are reduced herein, which may improve overall yields over the yields of previous methods.

Examples provided herein are related to modified nucleoside triphosphates, and methods for synthesizing modified nucleoside triphosphates.

### TERMS

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but can include additional steps. When used in the context of a compound, composition, or device, the term “comprising” means that the compound, composition, or device includes at least the recited features or components, but can also include additional features or components.

The terms “substantially”, “approximately”, and “about” used throughout this Specification are used to describe and account for small fluctuations, such as due to variations in processing. For example, they can refer to less than or equal to +5%, such as less than or equal to +2%, such as less than or equal to +1%, such as less than or equal to +0.5%, such as less than or equal to +0.2%, such as less than or equal to +0.1%, such as less than or equal to +0.05%.

As used herein, terms such as “covalently coupled” or “covalently bonded” refer to the forming of a chemical bond that is characterized by the sharing of pairs of electrons between atoms. For example, a covalently coupled molecule refers to a molecule that forms chemical bonds with a substrate, as compared to coupling to the surface via other means, for example, a non-covalent bond such as electrostatic interaction.

The term “halogen” or “halo,” as used herein, means fluorine, chlorine, bromine, or iodine, with fluorine and chlorine being examples.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group may be designated as “C₁₋₄ alkyl” or similar designations. By way of example only, “C₁₋₄ alkyl” or “C₁₋₄alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group may be designated as “C₂₋₄ alkenyl” or similar designations. By way of example only, “C₂₋₄ alkenyl” indicates that there are two to four carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, and hexenyl, and the like.

Groups that include an alkenyl group include optionally substituted alkenyl, cycloalkenyl, and heterocycloalkenyl groups.

As used herein, “alkynyl” refers to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 4 carbon atoms. The alkynyl group may be designated as “C₂₋₄ alkynyl” or similar designations. By way of example only, “C₂₋₄ alkynyl” or “C₂₋₄alkynyl” indicates that there are two to four carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

Groups that include an alkynyl group include optionally substituted alkynyl, cycloalkynyl, and heterocycloalkynyl groups.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some examples, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl.

As used herein, “heterocycle” refers to a cyclic compound which includes atoms of carbon along with another atom (heteroatom), for example nitrogen, oxygen or sulfur. Heterocycles may be aromatic (heteroaryl) or aliphatic. An aliphatic heterocycle may be completely saturated or may contain one or more or two or more double bonds, for example the heterocycle may be a heterocycloalkyl. The heterocycle may include a single heterocyclic ring or multiple heterocyclic rings that are fused.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some examples, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

As used herein, “cycloalkyl” means a fully saturated carbocyclyl ring or ring system. Examples include cyclopropyl, cyclobutyl, cyclopentyl, and cyclohexyl.

As used herein, “cycloalkenyl” or “cycloalkene” means a carbocyclyl ring or ring system having at least one double bond, wherein no ring in the ring system is aromatic. An example is cyclohexenyl or cyclohexene. Another example is norbornene or norbornenyl.

As used herein, “heterocycloalkenyl” or “heterocycloalkene” means a carbocyclyl ring or ring system with at least one heteroatom in ring backbone, having at least one double bond, wherein no ring in the ring system is aromatic. In some examples, heterocycloalkenyl or heterocycloalkene ring or ring system is 3-membered, 4-membered, 5-membered, 6-membered, 7-membered, 8-membered, 9-membered, or 10-membered.

As used herein, “cycloalkynyl” or “cycloalkyne” means a carbocyclyl ring or ring system having at least one triple bond, wherein no ring in the ring system is aromatic. An example is cyclooctyne. Another example is bicyclononyne. Another example is dibenzocyclooctyne (DBCO).

As used herein, “heterocycloalkynyl” or “heterocycloalkyne” means a carbocyclyl ring or ring system with at least one heteroatom in ring backbone, having at least one triple bond, wherein no ring in the ring system is aromatic. In some examples, heterocycloalkynyl or heterocycloalkyne ring or ring system is 3-membered, 4-membered, 5-membered, 6-membered, 7-membered, 8-membered, 9-membered, or 10-membered.

As used herein, “heterocycloalkyl” means a non-aromatic cyclic ring or ring system containing at least one heteroatom in the ring backbone. Heterocycloalkyls may be joined together in a fused, bridged or spiro-connected fashion. Heterocycloalkyls may have any degree of saturation provided that at least one heterocyclic ring in the ring system is not aromatic. The heterocycloalkyl group may have 3 to 20 ring members (i.e., the number of atoms making up the ring backbone, including carbon atoms and heteroatoms), although the present definition also covers the occurrence of the term “heterocycloalkyl” where no numerical range is designated. The heterocycloalkyl group may also be a medium size heterocycloalkyl having 3 to 10 ring members. The heterocycloalkyl group could also be a heterocycloalkyl having 3 to 6 ring members. The heterocycloalkyl group may be designated as “3-6 membered heterocycloalkyl” or similar designations. In some six membered monocyclic heterocycloalkyls, the heteroatom(s) are selected from one up to three of O, N or S, and in some five membered monocyclic heterocycloalkyls, the heteroatom(s) are selected from one or two heteroatoms selected from O, N, or S. Examples of heterocycloalkyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

As used herein, the term “nucleoside” is intended to mean a molecule that includes a sugar and at least one phosphate group, and in some examples also includes a nucleobase. A nucleoside that lacks a nucleobase can be referred to as “abasic.” Nucleosides include deoxyribonucleosides, modified deoxyribonucleosides, ribonucleosides, modified ribonucleosides, peptide nucleosides, modified peptide nucleosides, modified phosphate sugar backbone nucleosides, and mixtures thereof. Examples of nucleosides include adenosine, thymidine, cytidine, guanosine, uridine, deoxyadenosine, deoxythymidine, deoxycytidine, deoxyguanosine, and deoxyuridine.

As used herein, the term “nucleoside” also is intended to encompass any nucleoside analogue which is a type of nucleoside that includes a modified nucleobase and/or sugar compared to naturally occurring nucleosides. Example modified nucleobases include inosine, xanthine, hypoxanthine, 5-methylcytosine, 5-hydroxymethyl cytosine, 2-aminoadenine, 6-methyl adenine, 6-methyl guanine, 4-thiouracil, 8-hydroxyl adenine or guanine, 7-methylguanine, 7-methyladenine, 8-azaguanine, 8-azaadenine, or the like. Example modified nucleobases also include isocytosine, isoguanine, 2-aminopurine 2-propyl guanine, 2-propyl adenine, 2-thiouracil, 2-thiothymine, 2-thiocytosine, 5-thiouracil, 5-halocytosine, 5-propynyl uracil, 5-propynyl cytosine, 6-azo uracil, 6-azo cytosine, 6-azo thymine, 5-thiouracil, 8-halo adenine or guanine, 8-amino adenine or guanine, 8-thiol adenine or guanine, 8-thioalkyl adenine or guanine 5-halo substituted uracil or cytosine, 7-deazaguanine, 7-deazaadenine, 3-deazaguanine, 3-deazaadenine or the like. As used herein, the term “nucleotide” is intended to mean a molecule that includes a sugar and at least one of: a phosphate group, a phosphoramidate, and a phosphorothioate. In some examples a nucleotide also includes a nucleobase. A nucleotide that lacks a nucleobase can be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides adenosine triphosphate (ATP), thymidine triphosphate (TTP), cytidine triphosphate (CTP), guanosine triphosphate (GTP), uridine triphosphate (UTP), deoxyadenosine triphosphate (dATP), deoxythymidine triphosphate (dTTP), deoxycytidine triphosphate (dCTP), deoxyguanosine triphosphate (dGTP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xanthine, hypoxanthine, 5-methylcytosine, 5-hydroxymethyl cytosine, 2-aminoadenine, 6-methyl adenine, 6-methyl guanine, 4-thiouracil, 8-hydroxyl adenine or guanine, 7-methylguanine, 7-methyladenine, 8-azaguanine, 8-azaadenine, or the like. Example modified nucleobases also include isocytosine, isoguanine, 2-aminopurine 2-propyl guanine, 2-propyl adenine, 2-thiouracil, 2-thiothymine, 2-thiocytosine, 5-thiouracil, 5-halocytosine, 5-propynyl uracil, 5-propynyl cytosine, 6-azo uracil, 6-azo cytosine, 6-azo thymine, 5-thiouracil, 8-halo adenine or guanine, 8-amino adenine or guanine, 8-thiol adenine or guanine, 8-thioalkyl adenine or guanine 5-halo substituted uracil or cytosine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides can include any suitable number of phosphates, e.g., three, four, five, six, or more than six phosphates.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), locked nucleic acid (LNA), and analogues thereof. A polynucleotide can be a single stranded sequence of nucleotides, such as RNA or single stranded DNA, a double stranded sequence of nucleotides, such as double stranded DNA, DNA that is folded to form a hairpin that is partially single stranded and partially double stranded, double-stranded amalgamations in which there are molecules that are non-covalently coupled to one another (e.g., via reversible hydrogen binding), and/or can include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides can include non-naturally occurring DNA, such as enantiomeric DNA. The precise sequence of nucleotides in a polynucleotide can be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primer and a single stranded target polynucleotide, and can sequentially add nucleotides to the growing primer to form a “complementary copy” polynucleotide having a sequence that is complementary to that of the target polynucleotide. DNA polymerases may bind to the target polynucleotide and then move down the target polynucleotide sequentially adding nucleotides to the free hydroxyl group at the 3′ end of a growing polynucleotide strand. DNA polymerases may synthesize complementary DNA molecules from DNA templates. RNA polymerases may synthesize RNA molecules from DNA templates (transcription). Other RNA polymerases, such as reverse transcriptases, may synthesize cDNA molecules from RNA templates. Still other RNA polymerases may synthesize RNA molecules from RNA templates, such as RdRP. Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase.

Example DNA polymerases include Bst DNA polymerase, 9° Nm DNA polymerase, Phi29 DNA polymerase, DNA polymerase I (E. coli), DNA polymerase I (Large), (Klenow) fragment, Klenow fragment (3′-5′ exo-), T4 DNA polymerase, T7 DNA polymerase, Deep VentR™ (exo-) DNA polymerase, Deep VentR™ DNA polymerase, DyNAzyme™ EXT DNA, DyNAzyme™ II Hot Start DNA Polymerase, Phusion™ High-Fidelity DNA Polymerase, Therminator™ DNA Polymerase, Therminator™ II DNA Polymerase, VentR® DNA Polymerase, VentR® (exo-) DNA Polymerase, RepliPHI™ Phi29 DNA Polymerase, rBst DNA Polymerase, rBst DNA Polymerase (Large), Fragment (IsoTherm™ DNA Polymerase), MasterAmp™ AmpliTherm™, DNA Polymerase, Taq DNA polymerase, Tth DNA polymerase, Tfl DNA polymerase, Tgo DNA polymerase, SP6 DNA polymerase, Tbr DNA polymerase, DNA polymerase Beta, ThermoPhi DNA polymerase, and Isopol™ SD+ polymerase. In specific, nonlimiting examples, the polymerase is selected from a group consisting of Bst, Bsu, and Phi29. Some polymerases have an activity that degrades the strand behind them (3′ exonuclease activity). Some useful polymerases have been modified, either by mutation or otherwise, to reduce or eliminate 3′ and/or 5′ exonuclease activity.

Example RNA polymerases include RdRps (RNA dependent, RNA polymerases) that catalyze the synthesis of the RNA strand complementary to a given RNA template. Example RdRps include polioviral 3Dpol, vesicular stomatitis virus L, and hepatitis C virus NS5B protein. Example RNA Reverse Transcriptases. A non-limiting example list to include are reverse transcriptases derived from Avian Myelomatosis Virus (AMV), Murine Moloney Leukemia Virus (MMLV) and/or the Human Immunodeficiency Virus (HIV), telomerase reverse transcriptases such as (hTERT), SuperScript™ III, SuperScript™ IV Reverse Transcriptase, ProtoScript® II Reverse Transcriptase.

The terms “polynucleotide” and “oligonucleotide” are used interchangeably herein. The different terms are not intended to denote any particular difference in size, sequence, or other property unless specifically indicated otherwise. For clarity of description the terms can be used to distinguish one species of polynucleotide from another when describing a particular method or composition that includes several polynucleotide species.

As used herein, the term “polymer” refers to a molecule including many repeated subunits or recurring units. Non-limiting examples of polymer structures include linear, branched, or hyper-branched polymers. Polymers as described herein can be linear, branched, hyper-branched or dendritic. Different classes of polymer backbones include, but are not limited to, polyacrylamides, polyacrylates, polyurethanes, polysiloxanes, silicones, polyacroleins, polyphosphazenes, polyisocyanates, poly-ols, polysaccharides, polypeptides, and combinations thereof. A polymer can include one or more moieties that can react with one or more other moieties to form a covalent bond.

All ranges may include the upper and lower values, and all ranges and ratio limits disclosed herein may be combined. It is to be understood that unless specifically stated otherwise, references to “a,” “an,” and/or “the” may include one or more than one and that reference to an item in the singular may also include the item in the plural. Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item. Further, reference to, e.g., a “first” item and a “second” item does not mean that there are no intervening items, and such intervening items may be present.

Nucleoside Triphosphates with Modified Phosphate Chains and Methods of Synthesizing the Same

As will be discussed below, some nucleoside triphosphate analogues include a sugar, a nucleobase coupled to the sugar, a triphosphate group coupled to the sugar, a heteroatom coupled to an alpha phosphate of the triphosphate group, and a first substituent coupled to the heteroatom.

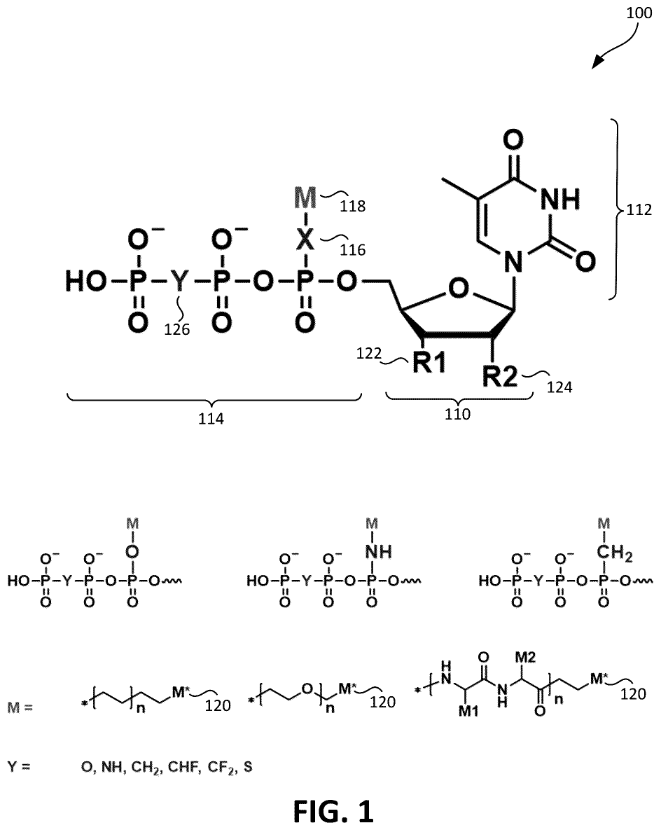

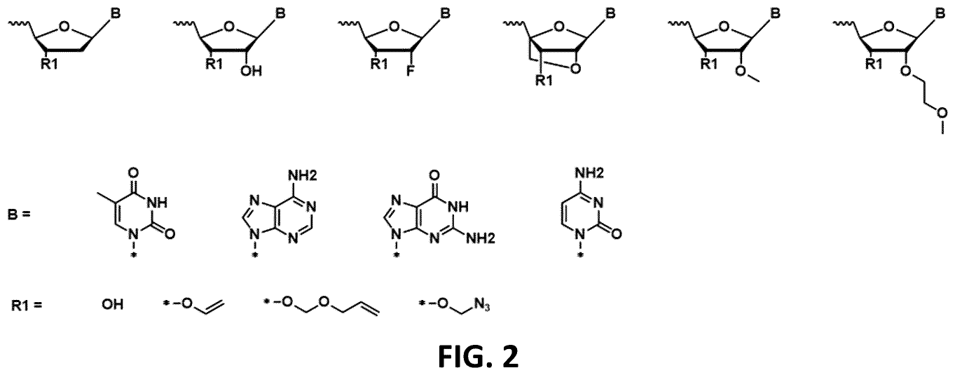

FIG. 1 illustrates example nucleoside triphosphate analogues provided herein, and FIG. 2 illustrates example nucleobases and sugar modifications that may be used in the nucleoside triphosphate analogues, respectively. Referring now to FIG. 1, nucleoside triphosphate analogue 100 may include sugar 110, nucleobase 112, triphosphate group 114, heteroatom 116, and first substituent 118. Nucleobase 112 may be bound to sugar 110. Triphosphate group 114 may be coupled to sugar 110. Heteroatom 116 may be coupled to an alpha phosphate of triphosphate group 114. First substituent 118 may be coupled to heteroatom 116.

In various examples, sugar 110 may include a five-carbon sugar. Illustratively, sugar 110 may include a natural sugar, such as ribose or deoxyribose. In other examples, sugar 110 may include a six carbon sugar, e.g., pyranose. Optionally, sugar 110 is a non-naturally occurring sugar, such as threose (as in threose nucleic acid, TNA). In various examples, sugar 110 may include an acyclic sugar moiety, e.g., may include glycerol (as in glycol nucleic acid, GNA). In various examples, sugar 110 may include a fluorine atom bound to the 2′ carbon of the sugar. Note that sugar 110 may be, but not necessarily be, cyclic. From the examples herein, it may be understood that sugar 110 illustratively may be a three-carbon sugar, a four-carbon sugar, a five-carbon sugar, or a six carbon sugar, and may be cyclic or acyclic.

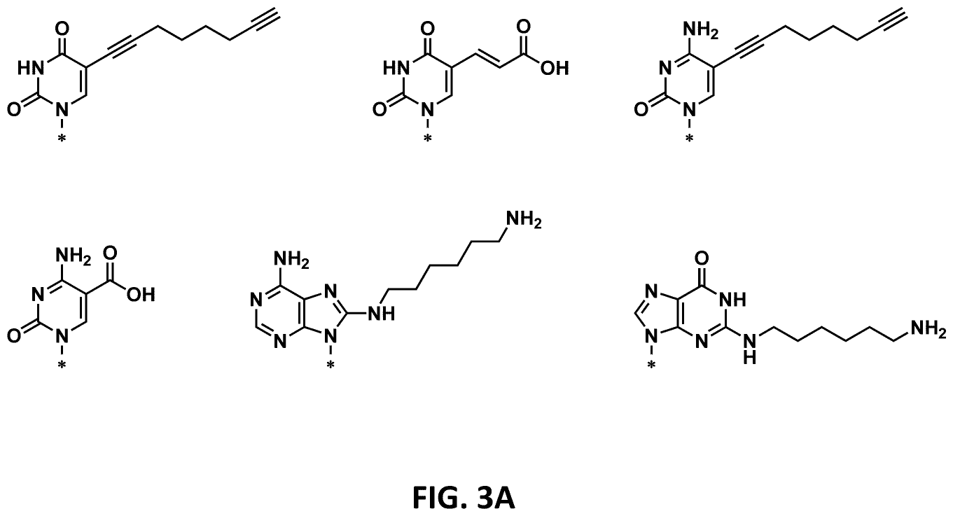

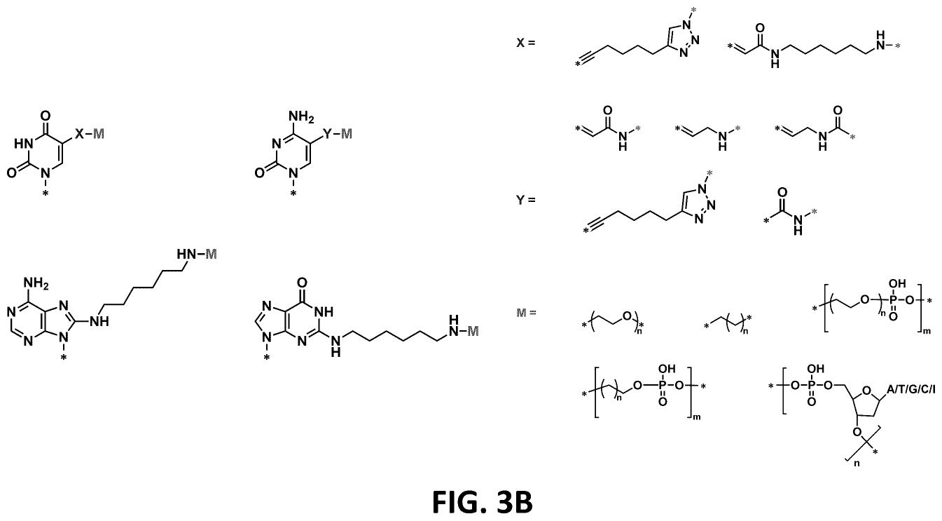

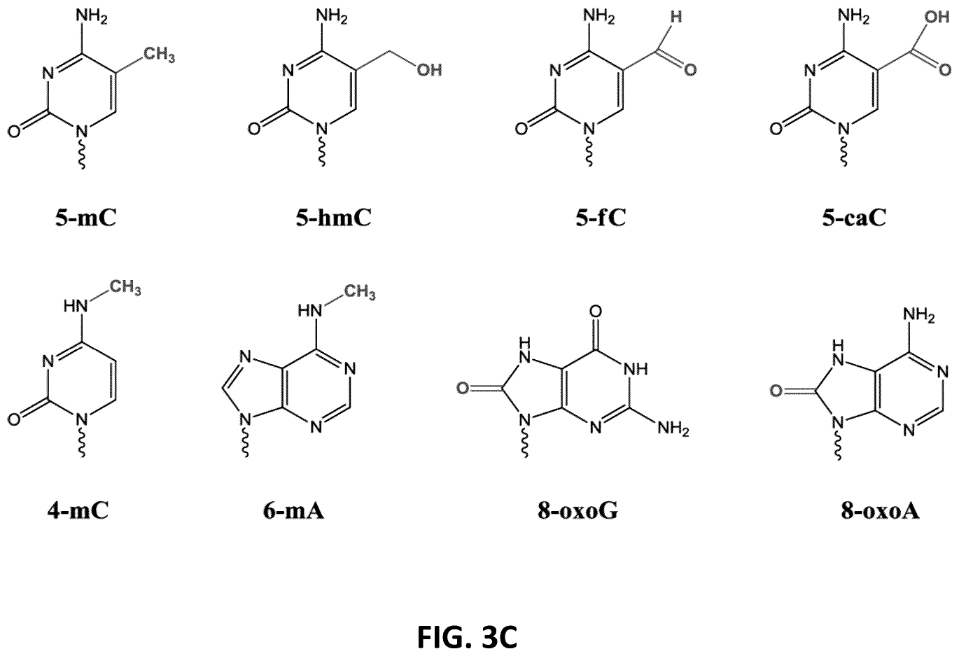

In various examples, as illustrated in FIG. 2, nucleobase 112 may include a naturally occurring nucleobase such as adenine, cytosine, guanine, thymine, or uracil. As illustrated in FIG. 3C, nucleobase 112 may include 5-methylcytosine, 5-hydroxymethylcytosine, 5-formylcytosine, 5-carboxylcytosine, 4-methylcytosine, 6-methyladenine, 8-oxoguanine, or 8-oxoadenine. Nucleobase 112 may include inosine, xanthine, hypoxanthine, 2-aminoadenine, 6-methyl guanine, 4-thiouracil, 8-hydroxyl adenine or guanine, 7-methylguanine, 7-methyladenine, 8-azaguanine, 8-azaadenine, or the like.

In other examples, as illustrated in FIG. 3A, nucleobase 112 may include a non-naturally occurring nucleobase, such as 5-(1,6-heptadiynyl)uracil, 5-(2-carboxyvinyl)uracil, 5-(1,6-heptadiynyl)cytosine, 5-carboxycytosine, 8-(1,6-diaminohexanyl)adenine, and 2-(1,6-diaminohexanyl)guanine. As illustrated in FIG. 3B, nucleobase 112 may include 5-(7-(1,2,3-triazole)hept-1-ynyl)uracil, 5-(N-(6-aminohexyl)acrylamide)uracil, 5-(methylacrylamido)uracil, 5-(N-allylmethylamino)uracil, 5-(N-allylacetamidyl)uracil, 5-(7-(1,2,3-triazole)hept-1-ynyl)cytosine, and 5-(methylacetamido)cytosine, 8-(1,6-diaminohexanyl)adenine, and 2-(1,6-diaminohexanyl)guanine. Each example of nucleobase 112 in the previous sentence may be further modified to include an ethylene oxide polymer, a polymethylene polymer, an (ethylene oxide) phosphate polymer, a polymethylene phosphate polymer, or a DNA polymer, as illustrated in FIG. 3B. Nucleobase 112 may include isocytosine, isoguanine, 2-aminopurine, 2-propyl guanine, 2-propyl adenine, 2-thiouracil, 2-thiothymine, 2-thiocytosine, 5-thiouracil, 5-halocytosine, 5-propynyl uracil, 5-propynyl cytosine, 6-azo uracil, 6-azo cytosine, 6-azo thymine, 5-thiouracil, 8-halo adenine or guanine, 8-amino adenine or guanine, 8-thiol adenine or guanine, 8-thioalkyl adenine or guanine 5-halo substituted uracil or cytosine, 7-deazaguanine, 7-deazaadenine, 3-deazaguanine, 3-deazaadenine or the like.

Referring back to FIG. 1, in various examples, heteroatom 116 may include an oxygen atom, a nitrogen atom, or a carbon atom. Without wishing to be bound by any theory, heteroatom 116 may alter the high-energy phosphoric anhydride bond, increasing the stability thereof and reducing the rate of hydrolysis. Additionally, or alternatively, heteroatom 116 may alter the rate of incorporation of nucleobase 112 by a polymerase. Heteroatom 116 may serve as a linker between triphosphate group 114 and first substituent 118.

In various examples, first substituent 118 may include a fluorescent dye, an alkyl chain, or a polymer. First substituent 118 may assist in the identification of nucleoside triphosphate analogue 110 in a sequencing application. Nonlimiting examples of polymers suitable for use in first substituent 118 (and other similar substituents coupled to the alpha phosphate such as described elsewhere herein) include a synthetic peptide or a synthetic polymer. In one nonlimiting example of a synthetic polymer, first substituent 118 (or other similar substituent coupled to the alpha phosphate such as described elsewhere herein) may include an ethylene glycol polymer, which may be referred to as poly(ethylene glycol). In various examples in which first substituent 118 includes a polymer, the polymer may include about 1 to about 50 repeating units, illustratively about 1 to about 10 repeating units, or about 11 to about 20 repeating units, or about 21 to about 50 repeating units.

In various examples illustrated in FIG. 1, first substituent 118 of nucleoside triphosphate analogue 100 may include functional group 120. Functional group 120 may assist in the identification of nucleoside triphosphate analogue 110 in a sequencing application. For example, the reaction of functional group 120 (M* in FIG. 1) may assist in the identification of nucleoside triphosphate analogue 110. Functional group 120 may include, illustratively, an amine, an azide, a carboxylic acid, a thiol, a tetrazine, a cyclooctyne, or an alkyne.

In various examples, nucleoside triphosphate analogue 100 may include second substituent 122 which may be coupled to the 3′ of sugar 110. In some examples, second substituent 122 may act as, or may include, a protecting group. In some examples, the protecting group of second substituent 122 may be used to mask the nucleophilic 3′-OH that would otherwise be reactive towards the chemistry used for the introduction of the phosphate group in a manner such as described further below. After the triphosphate group is installed on the nucleotide, the 3′-OH protecting group may be removed, for example via selective chemical treatment. For example, the protecting group TBDPS may be deprotected using TBAF, HF-TEA, or HF-pyridine. Second substituent 122 may, in some examples, act as a reversible terminator in a sequencing-by-synthesis (SBS) context. For example, in the SBS context, the 3′-group may be cleavable by selective methods depending on the identity. For example, if the 3′ group is a 3′-azido methyl group which protects a polymerase from adding another nucleotide to the 3′-end of the growing strand, treatment with TCEP or THP may be used to deprotect the 3′-azido methyl group and expose the 3′-OH so that the polymerase may then add another nucleotide to the 3′-OH. Second substituent 122 may be coupled to sugar 110 via a 3′ carbon of sugar 110. In nonlimiting examples such as illustrated in FIG. 2, second substituent 122 may include a vinyl group, an allyloxymethyl group, or an azido methyl group.

In various examples, nucleoside triphosphate analogue 100 may include third substituent 124 which may be coupled to the 2′ carbon of sugar 110. Third substituent 124 may act as a protecting group. Third substituent 124 may be a protecting group for an RNA synthesis and/or a substituent such as fluorine atom or a methoxy group as an RNA analogue. Additionally, or alternatively, third substituent 124 may be selected so as to increase the temperature at which a duplex including the nucleoside triphosphate analogue will dissociate. That is, the third substituent 124 may increase the melting temperature (Tm) of a duplex including the nucleoside triphosphate analogue. Third substituent 124 additionally, or alternatively, may improve resistance to a nuclease of a polynucleotide that includes the nucleotide triphosphate analogue. In non-limiting examples such as illustrated in FIG. 2, third substituent 124 may include a fluorine, a methoxy group, or an ethoxy group.

In various examples, nucleoside triphosphate analogue 100 may include fourth substituent 126 which may be coupled between a beta phosphate and a gamma phosphate of triphosphate group 114. Without wishing to be bound by any theory, it is believed that fourth substituent 126 may affect the stability of nucleoside triphosphate analogue 110. Illustratively, and without wishing to be bound by any theory, it is believed that the presence of a modification on the alpha phosphate may alter the electron density across the phosphate groups, resulting in instability, and that the fourth substituent may potentially rebalance the electron density to regain sufficient stability of the triphosphate towards degradation (loss of phosphate groups). In nonlimiting examples such as illustrated in FIG. 1B, fourth substituent 126 may include an oxygen atom, an amine, a methylene, a fluoromethylene, a difluoromethylene, a sulfur atom, or a selenium atom.

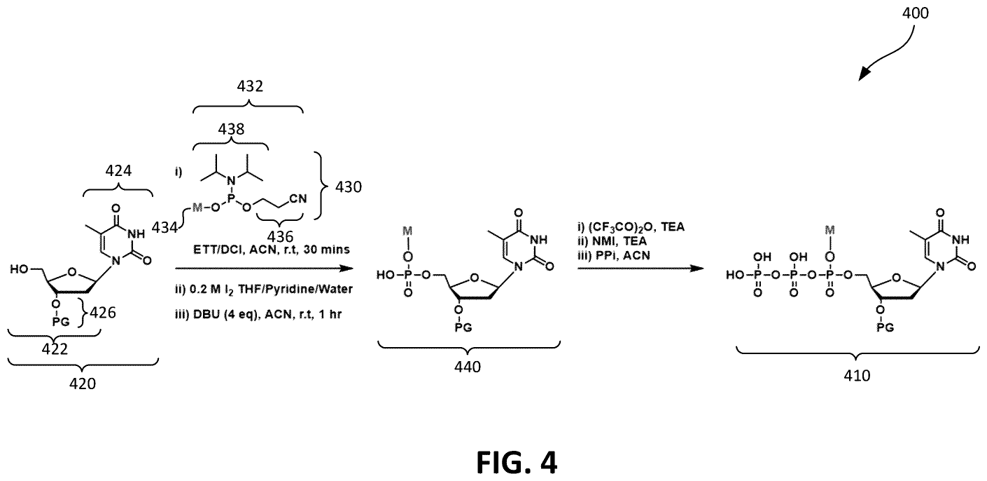

As will be described with reference to FIG. 4, a nucleoside triphosphate analogue may be synthesized beginning with a nucleoside. A phosphorylating agent comprising a protecting group may be coupled to the nucleoside, e.g., to a sugar of the nucleoside. The phosphorylating agent may be oxidized. After oxidation, the protecting group may be removed from the phosphorylating agent to form a monophosphate diester. The monophosphate diester may be activated. The monophosphate diester may be reacted with a pyrophosphate to form a triphosphate group. In some examples, the phosphorylating agent may include a substituent. As such, the monophosphate diester and the triphosphate group also may include the substituent.

FIG. 4 illustrates an example method of synthesizing a nucleoside triphosphate analogue. Referring now to FIG. 4, method 400 of synthesizing nucleoside triphosphate analogue 410 may include coupling phosphorylating agent 430 to nucleoside 420. Nucleoside 420 may include sugar 422 and nucleobase 424. Phosphorylating agent 430 may react with nucleoside 420 to couple a substituent 434 to nucleoside 420 via a heteroatom (here, oxygen) coupled to an alpha phosphate group.

Although the nonlimiting example in FIG. 4 includes deoxyribose, sugar 422 may include any suitable sugar, such as those described with reference to FIG. 1 or elsewhere herein. In various examples, sugar 422 may include protected 3′-O group 426 (O-PG in FIG. 4). Protected 3′-O group 426 may inhibit the 3′ oxygen of sugar 422 from being oxidized when phosphorylating agent 430 is oxidized in a manner such as will be described. Sugar 422 may include CH₂—OH at its 5′ position.

Phosphorylating agent 430 may include protecting group 432. Protecting group 432 may be converted into a leaving group, for example by being protonated by the activator. Illustratively, the diisopropyl amino group of amidite may be protonated by the activator, and thus converted into a good leaving group. In the nonlimiting example illustrated in FIG. 4, protecting group 432 may include propionitrile 436.

Although the nonlimiting example in FIG. 4 includes thymine, nucleobase 424 may include any suitable naturally occurring nucleobase, or any suitable non-naturally occurring nucleobase, such as those described with reference to FIG. 1 or elsewhere herein.

In the nonlimiting example illustrated in FIG. 4, phosphorylating agent 430 may include substituent 434 (denoted M in FIG. 4). Substituent 434 may assist in the identification of nucleoside triphosphate analogue 410 in a sequencing application. For example, substituent 434 may include any suitable fluorescent dye, alkyl chain, or polymer, such as discussed with reference to first substituent 118 in FIG. 1.

In the nonlimiting example illustrated in FIG. 4, phosphorylating agent 430 may include phosphoramidite 438. Phosphorylating agent 430 also or alternatively may include an N-diisopropyl group, a methyl group, an ethyl group, a propyl group, and/or a cyclohexyl group. The composition of phosphorylating agent 430 may change the reactivity of the P—N bond in phosphorylating agent 430. In various examples, phosphorylating agent 430 may be activated using 5-(ethylthio)-1H-tetrazole (ETT) and 4,5-dicyanoimidazole (DCI), tetrazole, or benzothiotetrazole. Activation of phosphorylating agent 430 may convert phosphorylating agent 430 into a leaving group, for example by protonating the diisopropylamino group of the phosphorylating agent. The protonated diisopropylamino group is then rapidly displaced by an attack of the 5′-hydroxyl group of the nucleoside on its neighbouring phosphorus atom, and a new phosphorus-oxygen bond is formed.

In various examples, method 400 may include oxidizing phosphorylating agent 430. Oxidizing phosphorylating agent 430 may make phosphorylating agent 430 more stable, for example by converting the oxidation state of phosphorylating agent 430 to P(V). Phosphorylating agent 430 may be oxidized using elemental iodine (I₂), t-butyl hydrogen peroxide (t-BuOOH), (1S)-(+)-(10-camphorsulfonyl)-oxaziridine (CSO), hydrogen peroxide, or meta-chloroperoxybenzoic acid (m-CPBA). Method 400 may include removing protecting group 432 from the phosphorylating agent 430 after phosphorylating agent 430 has been coupled to nucleoside 420, forming monophosphate diester 440. Forming monophosphate diester 440 may provide selectivity in creating an alpha-modified nucleotide triphosphate over a beta- or gamma-modified nucleotide triphosphate. In the nonlimiting example illustrated in FIG. 4, protecting group 432 may be removed using 1,8-diazabicyclo[5.4.0]undec-7-ene (DBU).

In various examples, method 400 may include activating monophosphate diester 440. Activating monophosphate diester 440 may convert monophosphate diester 440 into a leaving group, for example by protonating the monophosphate diester which is then rapidly displaced by the attack of the pyrophosphate on its neighboring phosphorus atom, and a new phosphorus-oxygen bond (triphosphate) is formed. In the nonlimiting example illustrated in FIG. 4, monophosphate diester 440 may be activated using trifluoroacetic anhydride (TFAA) and N-methylimidazole (NMI). Illustratively, monophosphate diester 440 may first be reacted with TFAA, be stirred for a time, and then be reacted with NMI. The reaction of monophosphate diester 440 with TFAA may include the reaction of TFAA with a hydroxyl of the monophosphate group of monophosphate diester 440 resulting in the addition of a trifluoroacetyl group to the monophosphate group of monophosphate diester 440. The subsequent reaction of monophosphate diester 440 with NMI may include the replacement of the trifluoroacetyl group with an NMI group, resulting in an activated monophosphate diester. As illustrated in FIG. 4, pyrophosphate then may be added to generate nucleotide triphosphate analogue 410 including substituent 434 coupled to the alpha-phosphate via an oxygen.

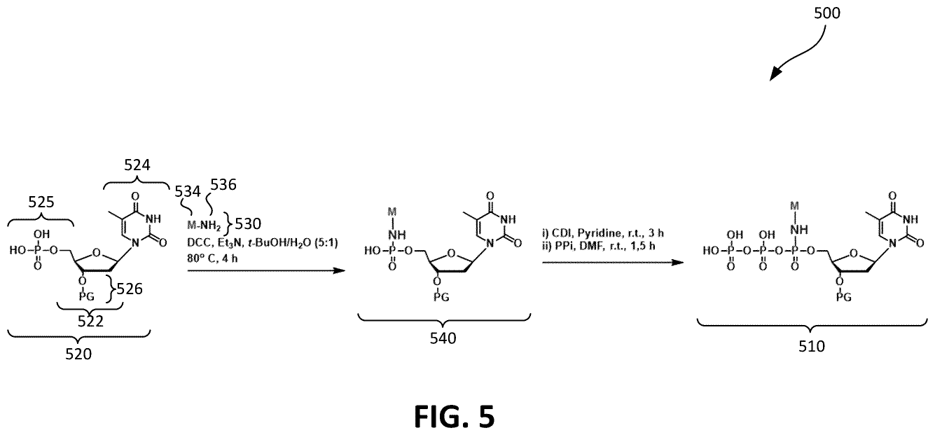

As will be described with reference to FIG. 5, a nucleoside triphosphate analogue may be synthesized beginning with a nucleotide. The nucleotide may be activated; for example, the nucleotide may include a monophosphate group which is activated. The nucleotide may be reacted with an amidation agent in an amidation reaction to form an amidated nucleotide, for example a nucleotide including an amidated monophosphate group. The amidated nucleotide may be activated, for example, by activating the amidated monophosphate group. The amidated nucleotide may be reacted with a pyrophosphate to form a triphosphate group. In some examples, the amidation agent may add a substituent to the monophosphate group, such that the triphosphate group also includes the substituent (at the alpha-phosphate position).

Referring now to FIG. 5, method 500 of synthesizing nucleoside triphosphate analogue 510 may include activating nucleotide 520. Nucleotide 520 may include sugar 522, nucleobase 524, and monophosphate group 525. Although the nonlimiting example in FIG. 5 includes deoxyribose, sugar 522 may include any suitable sugar, as described with reference to FIG. 1 or elsewhere herein. Sugar 522 may include protected 3′-O group 526 (O-PG in FIG. 5). Protected 3′-O group 526 may inhibit the 3′ oxygen of sugar 522 from being oxidized when phosphorylating agent 530 is oxidized in a manner such as will be discussed.

Although the nonlimiting example in FIG. 5 includes thymine, nucleobase 524 may include any suitable naturally occurring nucleobase, or any suitable non-naturally occurring nucleobase, as described with reference to FIG. 1 or elsewhere herein.

Method 500 may include reacting the monophosphate group of nucleotide 520 with amidation agent 530 in an amidation reaction to form amidated nucleotide 540. Amidation agent 530 may react with monophosphate group 525 to attach a heteroatom (here, nitrogen), which is attached to substituent 534, to monophosphate group 525.

Amidation agent 530 may include substituent 534. Substituent 534 may assist in the identification of nucleoside triphosphate analogue 510 in a sequencing application. For example, substituent 534 may include any suitable fluorescent dye, alkyl chain, or polymer, for example such as discussed with reference to first substituent 118 in FIG. 1 or elsewhere herein. In various examples, amidation agent 530 may include amine 536. In various examples, amine 536 may include a primary amine or a secondary amine. In various examples, amine 536 may include an alkyne, for example to be using in a click conjugation chemistry. In various examples, amidation agent 530 may include a carboxylic acid.

In various examples, method 500 may include activating amidated nucleotide 540, e.g., activating the amidated monophosphate group. In the nonlimiting example shown in FIG. 5, the amidated monophosphate group may be activated using carbonyldiimidazole (CDI) and pyridine. In some examples, the amidated monophosphate group may first be reacted with CDI and then may be reacted with pyridine. Alternatively, the amidated monophosphate group may be reacted concurrently with CDI and pyridine. The reaction with CDI may generate a phosphor imidazolinium intermediate and thus promote the triphosphate synthesis efficiently after adding pyrophosphate in a manner such as described below. The with pyridine may include the replacement of a hydroxyl group of the monophosphate group of the amidated nucleotide 540 with a pyridine, resulting in an activated amidated nucleotide.

Method 500 may include reacting the amidated nucleotide with a pyrophosphate to form a triphosphate group. Illustratively, the activated, amidated monophosphate group may be reacted with a pyrophosphate to form a triphosphate group. The triphosphate group may include substituent 534 (M) coupled to the alpha phosphate via a heteroatom (here, oxygen). In nonlimiting examples where amidated nucleotide 540 is reacted with CDI, CDI may promote the synthesis of the triphosphate group after the addition of the pyrophosphate.

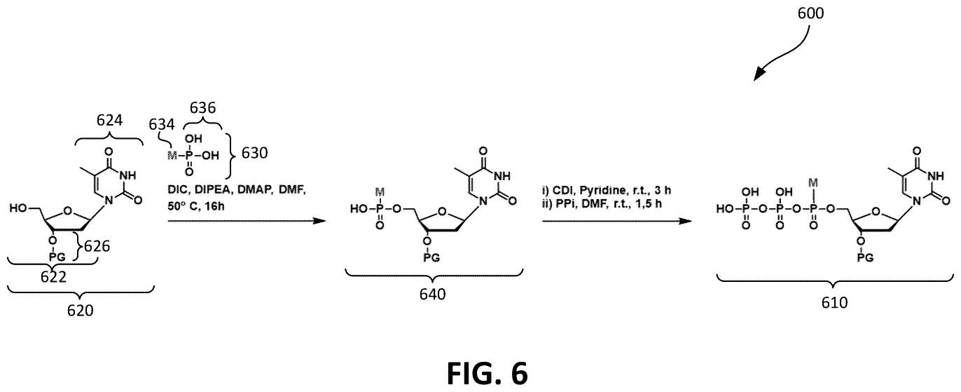

As will be described with reference to FIG. 6, a nucleoside triphosphate analogue may be synthesized using a phosphorylating agent. The phosphorylating agent may be activated. The activated phosphorylating agent may be coupled with a nucleoside (e.g., to a sugar of the nucleoside), forming a monophosphate diester. The monophosphate diester may be activated. The monophosphate diester may be reacted with a pyrophosphate to form a triphosphate group. In some examples, the phosphorylating agent may include a substituent. The substituent may be incorporated into the monophosphate diester and thus into the triphosphate group.

FIG. 6 illustrates another example method of synthesizing a nucleoside triphosphate analogue. Referring now to FIG. 6, method 600 of synthesizing nucleoside triphosphate analogue 610 may include activating phosphorylating agent 630. Phosphorylating agent 630 may react with nucleoside 620 to couple substituent 634 to nucleoside 620, e.g., to the alpha phosphate of the triphosphate group. Whereas examples such as described with reference to FIGS. 4 and 5 include a heteroatom coupling the substituent to the alpha phosphate of the triphosphate group, in the example shown in FIG. 6 the heteroatom may be directly coupled to the phosphorous atom of the alpha phosphate.

Phosphorylating agent 630 may include substituent 634. Substituent 634 may assist in the identification of nucleoside triphosphate analogue 610 in a sequencing application. For example, substituent 634 may include any suitable fluorescent dye, alkyl chain, or polymer, for example such as discussed with reference to first substituent 118 in FIG. 1.

In various examples, phosphorylating agent 630 may include phosphonate 636. In various examples, phosphorylating agent 630 may be activated using N,N′-diisopropylcarbodiimide (DIC), N,N′-diisopropylethylamine (DIPEA), and 4-dimethylaminopyridine (DMAP). In various examples, phosphorylating agent 630 may first be activated using DIC, for example by activating the monophosphate derivative. The activated phosphorylating agent may then be reacted with DIPEA and DMAP.

Method 600 may include coupling activated phosphorylating agent 630 with nucleoside 620, forming monophosphate diester 640. For example, the activated phosphorylating agent may be coupled to the nucleoside sugar's 5′-OH to make nucleoside phosphodiester. Nucleoside 620 may include sugar 622 and nucleobase 624.

Although the nonlimiting example in FIG. 6 includes deoxyribose, sugar 622 may include any suitable sugar, such as described with reference to FIG. 1 or elsewhere herein. Sugar 622 may include protected 3′-O group 626. Protected 3′-O group 626 may inhibit the 3′ oxygen of sugar 622 from being oxidized when phosphorylating agent 630 is activated in a manner such as will be discussed.

Although the nonlimiting example in FIG. 6 includes thymine, nucleobase 624 may include any suitable naturally occurring nucleobase, or any suitable non-naturally occurring nucleobase, such as described with reference to FIG. 1 or elsewhere herein. In various examples, method 600 may include activating monophosphate diester 640. Illustratively, monophosphate diester 640 may be activated and reacted, e.g., using carbonyldiimidazole (CDI) and pyridine, to generate a phospho-imidazolinium intermediate. Method 600 may include subsequently reacting monophosphate diester 640 with a pyrophosphate. In examples where monophosphate diester 640 is reacted with CDI, CDI may promote the synthesis of the triphosphate group after the addition of the pyrophosphate. The subsequent reaction of monophosphate diester 640 with a pyrophosphate may form a triphosphate group of nucleoside triphosphate analogue 610. In examples in which the phosphorylating agent includes a substituent, the monophosphate diester and the triphosphate group also may include the substituent (e.g., coupled to the alpha phosphate of the triphosphate group).

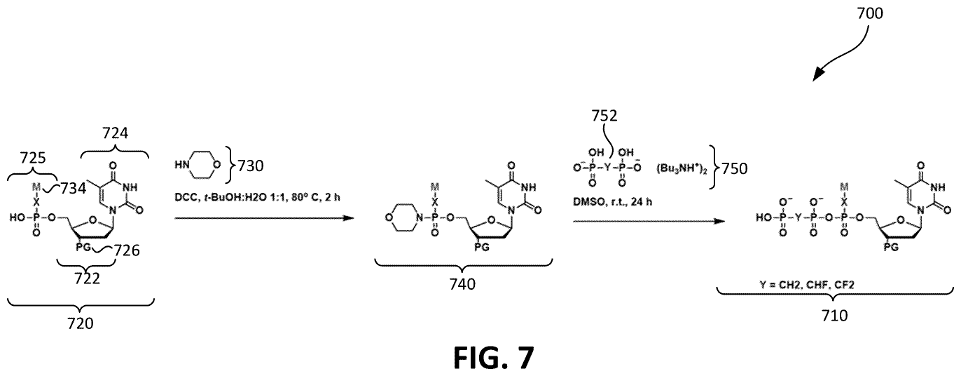

As will be described with reference to FIG. 7, a nucleoside triphosphate analogue may be synthesized using a nucleotide analogue. The monophosphate group of the nucleotide analogue may be reacted with a morpholidation agent to form a phosphormorpholidate group. The phosphormorpholidate group may be reacted with a bisphosphonic acid salt to form a triphosphate group. In some examples, the monophosphate group of the nucleoside triphosphate analogue may include a first substituent, such that the phosphormorpholidate group and the triphosphate group also include the first substituent (e.g., at an alpha phosphate of the triphosphate group). In this regard, the nucleoside triphosphate analogue may be prepared in a manner such as described with reference to FIG. 4, 5 or 6. Additionally, or alternatively, the bisphosphonic acid salt may include a second substituent (e.g., may be a modified bisphosphonic acid salt), such that the triphosphate group may include the second substituent between the beta phosphorous and the gamma phosphorous of the triphosphate group.

FIG. 7 illustrates another example method of synthesizing a nucleoside triphosphate. Referring now to FIG. 7, method 700 of synthesizing nucleoside triphosphate analogue 710 may include reacting nucleotide analogue 720 with morpholidation agent 730 to form phosphormorpholidate 740. For example, morpholidation agent 730 may react with the monophosphate group 725 of nucleotide analogue 720 to couple a morpholine group to the monophosphate group.

Nucleotide analogue 720 may include sugar 722, nucleobase 724, monophosphate group 725, and first substituent 734. Optionally, monophosphate group 725 includes substituent 734 which may be coupled directly to the phosphorous atom (e.g., in a manner such as described with reference to FIG. 6) or may be coupled to the phosphorous atom via a heteroatom (e.g., in a manner such as described with reference to FIG. 4 or 5).

Although the nonlimiting example in FIG. 7 includes deoxyribose, sugar 722 may include any suitable sugar, such as described with reference to FIG. 1 or elsewhere herein. Sugar 722 may include protecting group 726 (PG in FIG. 7) that is coupled to a 3′ carbon of sugar 722. Protecting group 726 may inhibit the 3′ carbon of sugar 722 from reacting when nucleotide analogue 720 is reacted with morpholidation agent 730 in a manner such as will be described.

Although the nonlimiting example in FIG. 7 includes thymine, nucleobase 724 may include any suitable naturally occurring nucleobase, or any suitable non-naturally occurring nucleobase, such as described with reference to FIG. 1 or elsewhere herein.

Substituent 734, which optionally is coupled to monophosphate group 725 directly or indirectly, may include any suitable fluorescent dye, alkyl chain, or polymer, such as discussed with reference to first substituent 118 in FIG. 1. Illustratively, substituent 734 may assist in the identification of nucleoside triphosphate analogue 710 in a sequencing application.

In various examples, morpholidation agent 730 may include morpholine. In various examples, the monophosphate 725 may be activated and then reacted with morpholidation agent 730 to form phosphomorpholidate 740 using a dehydrating agent, such as diisopropylcarbodiimide (DIC), 1-ethyl-3-(3-dimethylaminopropyl)carbodiimide (EDCI), benzotriazole-1-yloxytripyrrolidinophosphonium hexafluorophosphate (pyBOP), or (1-cyano-2-ethoxy-2-oxoethylidenaminooxy)dimethylamino-morpholino-carbenium hexafluorophosphate (COMU). In various examples, the resulting monophosphate derivative of the nucleotide analogue 720 may be activated using N,N′-dicyclohexylcarbodiimide (DCC). Such activation may promote the reaction of the 5′-OHgroup to form a nucleoside phosphodiester by adding, for example, DIPEA and DMAP.

In various examples, method 700 may include reacting phosphormorpholidate 740 with modified bisphosphonic acid salt 750 to form a triphosphate group. Optionally, in various examples, modified bisphosphonic acid salt 750 may include second substituent 752. Second substituent 752 may be coupled to each phosphate group of modified bisphosphonic acid salt 750. In nonlimiting examples such as illustrated in FIG. 7, second substituent 752 may include an oxygen atom, a nitrogen atom, a methylene, a fluoromethylene, a difluoromethylene, a sulfur atom, or a selenium atom. In such examples, second substituent 752 may be located between the beta phosphate and gamma phosphates of the triphosphate group. Without wishing to be bound by any theory, it is believed that second substituent 752 may affect the stability of nucleoside triphosphate analogue 710 by altering the electron flow or electron density across the triphosphate group in a manner that favors stabilization of the triphosphate group. Modified bisphosphonic acid salt 750 may include any suitable counterion, such as a tributylammonium counterion.

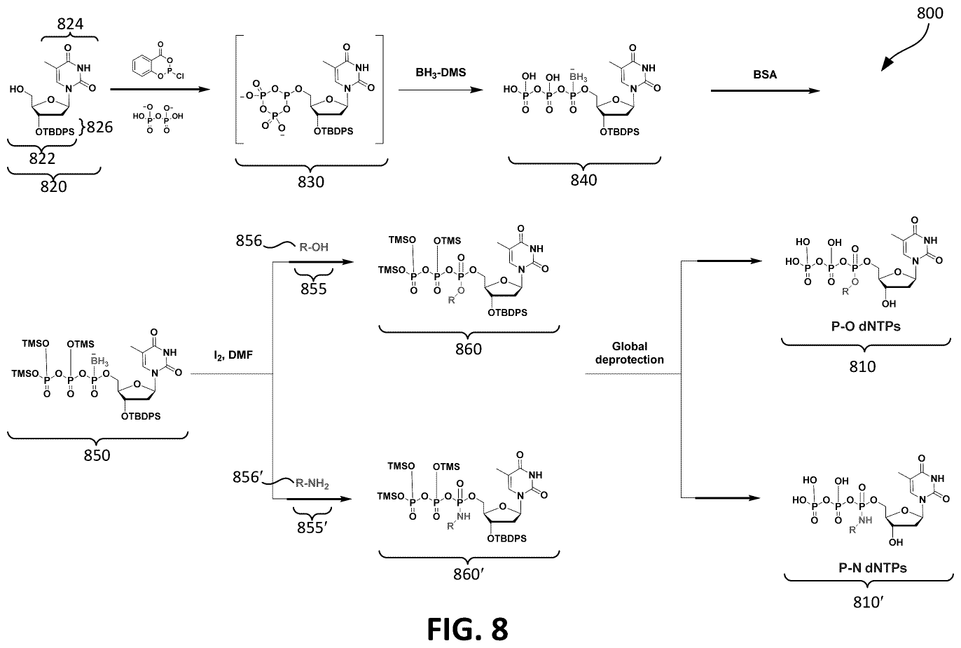

As will be described with reference to FIG. 8, a nucleoside triphosphate analogue may be synthesized using a protected nucleoside. The protected nucleoside may be reacted with a phosphite and a pyrophosphate to form a first intermediate. The first intermediate may be reduced using a borane to form a boranophosphate intermediate. The boranophosphate intermediate may be protected to form a protected boranophosphate intermediate. The protected boranophosphate intermediate may be activated and reacted with a reagent to form a second intermediate which includes a protected triphosphate group. The second intermediate may be deprotected to form a triphosphate group. In some examples, the reagent, with which the protected boranophosphate intermediate is reacted, includes a substituent. As such, the protected triphosphate group may include the substituent, and the triphosphate group may include the substituent. In some examples, the substituent may be coupled to the alpha phosphate of the triphosphate group, e.g., via a heteroatom.

FIG. 8 illustrates another example method of synthesizing a nucleoside triphosphate analogue. Referring now to FIG. 8, method 800 of synthesizing nucleoside triphosphate analogue 810 or 810′ may include reacting protected nucleoside 820 with a pyrophosphate to form first intermediate 830. Protected nucleoside 820 may include sugar 822, nucleobase 824, and protected 3′-O group 826. Although the nonlimiting example in FIG. 8 includes deoxyribose, sugar 822 may include any suitable sugar, such as described with reference to FIG. 1 or elsewhere herein. Additionally, although the nonlimiting example in FIG. 8 includes thymine, nucleobase 824 may include any suitable naturally occurring nucleobase, or any suitable non-naturally occurring nucleobase, for example such as described with reference to FIG. 1 or elsewhere herein. In the nonlimiting example illustrated in FIG. 8, protected 3′-O group 826 of protected nucleoside 820 may include tert-butyldiphenylsilyl (TBDPS). Protected 3′-O group 826 may inhibit the 3′ oxygen of sugar 822 from reacting with the pyrophosphate in a manner such as will be described.

In the nonlimiting example illustrated in FIG. 8, the 5′-OH group of the protected nucleoside 820 may be activated with salicyl chlorophosphite. Such activation may promote the reaction of the 5′-OH to form intermediate 830 including a cyclic triphosphate upon adding pyrophosphate. The cyclic triphosphate then may be hydrolyzed by water to generate a linear triphosphate (hydrolysis not specifically illustrated in FIG. 8).

In various examples, method 800 may include reducing first intermediate 830 to form boranophosphate intermediate 840. For example, first intermediate 830 may be reduced using a borane-containing reagent such as borane dimethylsulfide (BH₃-DMS, in the nonlimiting example shown in FIG. 8) or other suitable borane, such as borane diisopropylethylamine. In various examples, the borane-containing reagent may generate a borano-substitution at the alpha phosphate of first intermediate 830. Such a borano-substitution may change polarity of the alpha phosphate, for example increasing the electropositive character at a phosphorous with an oxidation level of P(V) and the nucleophilic handles attached to the phosphorous.

In various examples, method 800 may include protecting boranophosphate intermediate 840 to form protected boranophosphate intermediate 850. Protecting boranophosphate intermediate 840 and forming protected boranophosphate intermediate 850 may inhibit reagent 855 from attaching substituent 856 to a protected oxygen. Boranophosphate intermediate 840 may be protected using bis(trimethylsilyl)acetamide (BSA). Boranophosphate intermediate 840 may be protected using any appropriate silylating agent. Protected boranophosphate intermediate 850 may include, for example, a trimethylsilyl group (TMS).

In various examples, method 800 may include activating protected boranophosphate intermediate 850 as part of the process of forming second intermediate 860 or 860′. In various examples, protected boranophosphate intermediate 850 may be activated using elemental iodine, a mesylate, and/or a triflate. In various examples, protected boranophosphate intermediate 850 may undergo nucleophilic substitution with reagent 855 or 855′. Reagent 855 or 855′ may include substituent 856 or 856′, denoted as R in FIG. 8. In various examples, substituent 856 or 856′ may include any suitable fluorescent dye, alkyl chain, or polymer, for example such as discussed with reference to first substituent 118 in FIG. 1. Substituent 856 or 856′ may assist in the identification of nucleoside triphosphate analogue 810 or 810′ in a sequencing application. Reagent 855 may include an alcohol group, used to install an —OR group on the alpha phosphate of boranophosphate intermediate 850 and thus to generate second intermediate 860. Alternatively, reagent 855′ may include an amine group, used to install an —NH—R group on the alpha phosphate of boranophosphate intermediate 850 and thus to generate second intermediate 860′.

In various examples, method 800 may include deprotecting the second intermediate to generate dNTPs which are ready for use in SBS. For example, second intermediate 860 may be deprotected to generate P—O dNTPs 810, or second intermediate 860′ may be deprotected to generate P—N dNTPs.

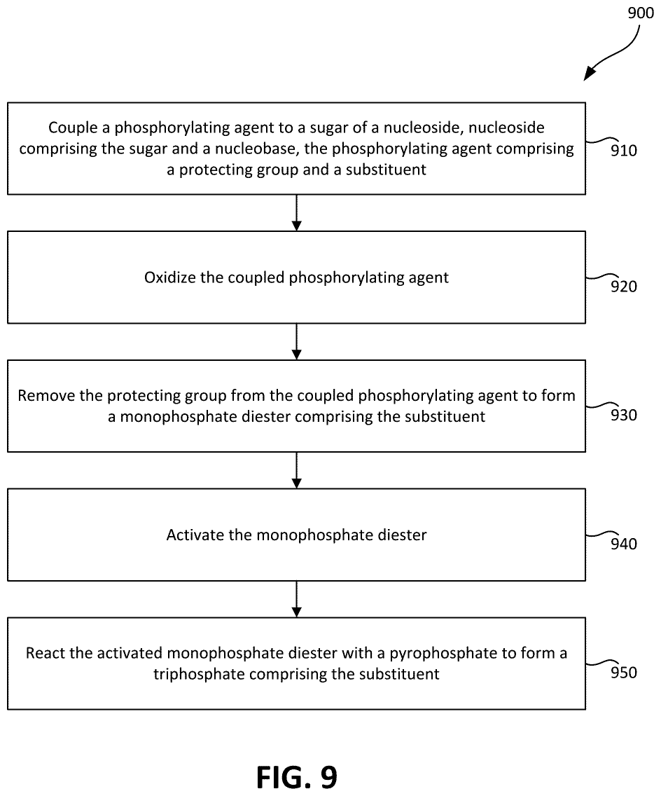

FIG. 9 illustrates a flow chart of operations in an example method of synthesizing a nucleoside triphosphate analogue. Referring now to FIG. 9, a phosphorylating agent may be coupled to a sugar of a nucleoside (operation 910). In a manner such as described with reference to FIG. 4, the nucleoside may include a sugar and a nucleobase. The sugar may include any suitable sugar, for example as described with reference to FIG. 1 or elsewhere herein. The sugar may include a protected 3′-O group. The nucleobase may include any suitable naturally occurring nucleobase, or any suitable non-naturally occurring nucleobase, such as described with reference to FIG. 1 or elsewhere herein. The phosphorylating agent may include a substituent. In some examples, the substituent may include any suitable fluorescent dye, alkyl chain, or polymer, for example as discussed with reference to first substituent 118 in FIG. 1. The phosphorylating agent also may include a protecting group. Nonlimiting examples of suitable protecting groups for phosphorylating agents are described with reference to FIG. 4.

With continued reference to FIG. 9, the coupled phosphorylating agent may then be oxidized (operation 920). In some examples, the phosphorylating agent may be oxidized using elemental iodine (I₂), t-butyl hydrogen peroxide (t-BuOOH), (1S)-(+)-(10-camphorsulfonyl)-oxaziridine (CSO), hydrogen peroxide, or meta-chloroperoxybenzoic acid (m-CPBA), e.g., as described with reference to FIG. 4.

With continued reference to FIG. 9, the protecting group may then be removed from the coupled phosphorylating agent to form a monophosphate diester (operation 930). In various examples, the protecting group may be removed using 1,8-diazabicyclo[5.4.0]undec-7-ene (DBU).

With continued reference to FIG. 9, the monophosphate diester may then be activated (operation 940), and the activated monophosphate diester may then be reacted with a pyrophosphate to form a triphosphate that includes the substituent (operation 950). Nonlimiting examples of reagents for activating the monophosphate diester are provided above with reference to FIG. 4.

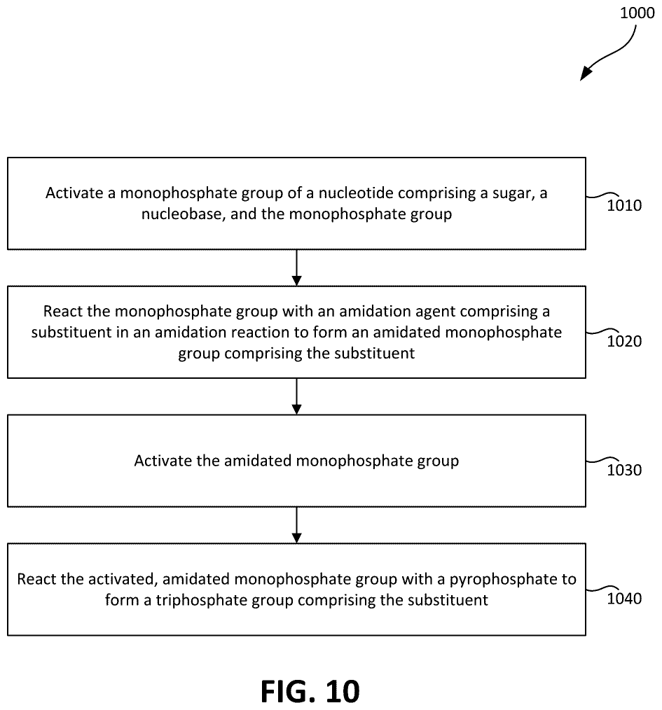

FIG. 10 illustrates a flow chart of operations in another example method of synthesizing a nucleoside triphosphate analogue. Referring now to FIG. 10, a monophosphate group of a nucleotide may be activated (operation 1010). In various examples, the nucleotide may include a sugar, a nucleobase, and the phosphate group. The sugar may include any suitable sugar, for example such as described with reference to FIG. 1 or elsewhere herein. The sugar may include a protected 3′-O group. The protected 3′-O group may inhibit the 3′-oxygen of the sugar from being oxidized when the phosphorylating agent is oxidized in a manner such as will be described. The nucleobase may include any suitable naturally occurring nucleobase, or any suitable non-naturally occurring nucleobase, for example such as described with reference to FIG. 1 or elsewhere herein.

With continued reference to FIG. 10, the nucleotide may then be reacted with an amidation agent that includes a substituent, in an amidation reaction, to form an amidated monophosphate group that includes the substituent (operation 1020). In various examples, the amidation agent may include an amine. In a manner such as described with reference to FIG. 5, the amidation agent may react with the nucleotide to attach a nitrogen heteroatom to the phosphorous atom, via which heteroatom the substituent may be attached to the nucleotide. In a manner such as described with reference to FIG. 1, the substituent may assist in the identification of the nucleoside triphosphate analogue in a sequencing application. The substituent may include any suitable fluorescent dye, alkyl chain, or polymer, such as discussed with reference to first substituent 118 in FIG. 1.

With continued reference to FIG. 10, the amidated monophosphate group may then be activated (operation 1030). Nonlimiting examples of reagents for activating an amidated monophosphate are described with reference to FIG. 5. With continued reference to FIG. 10, the activated, amidated monophosphate group may then be reacted with a pyrophosphate to form a triphosphate group that includes the substituent (operation 1040), e.g., in a manner such as described with reference to FIG. 5.

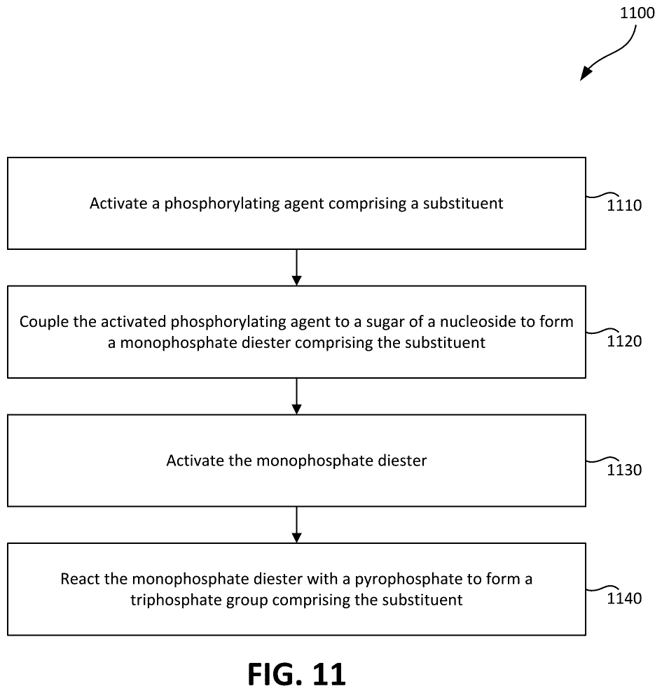

FIG. 11 illustrates a flow chart of operations in another example method of synthesizing a nucleoside triphosphate analogue. Referring now to FIG. 11, a phosphorylating agent may be activated that includes a substituent (operation 1110). Nonlimiting examples of phosphorylating agents, and reagents for activating a phosphorylating agent, are described with reference to FIG. 6. In a manner such as described with reference to FIG. 1, the substituent may assist in the identification of the nucleoside triphosphate analogue in a sequencing application. The substituent may include any suitable fluorescent dye, alkyl chain, or polymer, for example such as discussed with reference to first substituent 118 in FIG. 1.

With continued reference to FIG. 11, the activated phosphorylating agent may then be coupled to a sugar of a nucleoside to form a monophosphate diester that includes the substituent (operation 1120). In various examples, the nucleoside may include the sugar and a nucleobase. The sugar may include any suitable sugar, such as described with reference to FIG. 1 or elsewhere herein. The sugar may include a protected 3′-O group. The protected 3′-O group may inhibit the 3′ oxygen of the sugar from being oxidized when the phosphorylating agent is activated. The nucleobase may include any suitable naturally occurring nucleobase, or any suitable non-naturally occurring nucleobase, for example such as described with reference to FIG. 1 or elsewhere herein.

With continued reference to FIG. 11, the monophosphate diester may then be activated (operation 1130). Nonlimiting examples of reagents and procedures for activating a monophosphate diester are provided with reference to FIG. 6. With continued reference to FIG. 11, the activated monophosphate diester may then be reacted with a pyrophosphate to form a triphosphate group that includes the substituent (operation 1140), e.g., in a manner such as described with reference to FIG. 6.

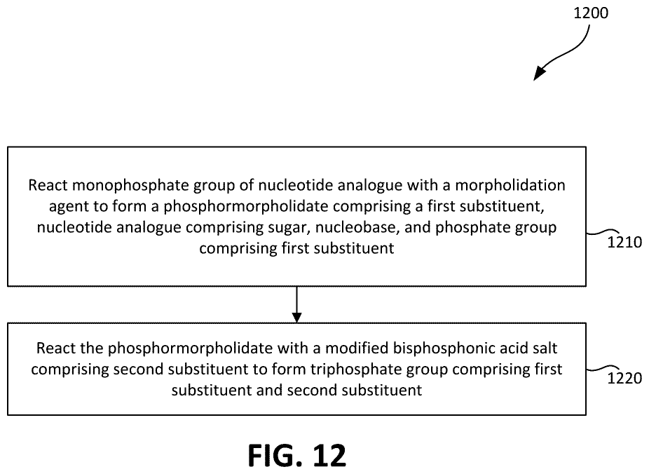

FIG. 12 illustrates a flow chart of operations in another example method of synthesizing a nucleoside triphosphate analogue. Referring now to FIG. 12, a method for synthesizing a nucleoside triphosphate analogue may include reacting a monophosphate group of a nucleotide analogue with a morpholidation agent to form a phosphormorpholidate (operation 1210). In various examples, the nucleotide analogue may include a first substituent, a sugar, a nucleobase, and the monophosphate group. As described with reference to FIG. 7, the monophosphate group may include a substituent. The substituent may be coupled to the monophosphate group via a heteroatom, e.g., in a manner such as described with reference to FIG. 4 or FIG. 5. Alternatively, the substituent may be coupled directly to the phosphorous of the monophosphate group in a manner such as described with reference to FIG. 6. The substituent may include any suitable fluorescent dye, alkyl chain, or polymer, such as discussed with reference to first substituent 118 in FIG. 1. The sugar may include any suitable sugar, for example such as described with reference to FIG. 1 or elsewhere herein. The sugar may include a protecting group coupled to a 3′ carbon of the sugar. The protecting group may inhibit the 3′ carbon of the sugar from reacting when the nucleotide analogue is reacted with the morpholidation agent. The nucleobase may include any suitable naturally occurring nucleobase, or any suitable non-naturally occurring nucleobase, for example, as described with reference to FIG. 1 or elsewhere herein. Nonlimiting examples of morpholidation agents, and reagents for coupling morpholidation agents to monophosphate groups, are described with reference to FIG. 7.

With continued reference to FIG. 12, the phosphormorpholidate may then be reacted with a modified bisphosphonic acid salt to form a triphosphate group which comprises the first substituent (operation 1220). Optionally, the modified bisphosphonic acid salt may include a second substituent, such that the triphosphate group which is formed in operation 1220 also includes the second substituent in a manner such as illustrated in FIG. 7. In examples in which the second substituent is used, the second substituent may be coupled to each phosphate group of the modified bisphosphonic acid salt. Optionally, the second substituent may include an oxygen atom, a nitrogen atom, a methylene, a fluoromethylene, a difluoromethylene, a sulfur atom, or a selenium atom. In an alternative configuration of FIG. 12, the monophosphate group of the nucleotide analogue does not include the first substituent, and is reacted with the morpholidation agent to form a phosphomorpholidate which does not include the first substituent. This phosphomorpholidate then is reacted with a modified bisphosphonic salt that does include the second substituent, to form a triphosphate group that includes the second substituent and does not include the first substituent.

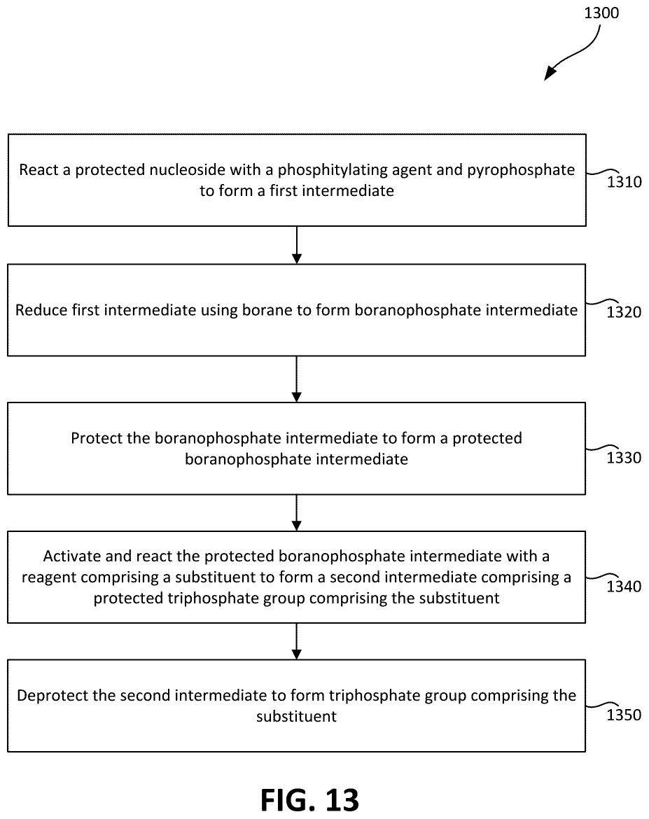

FIG. 13 illustrates a flow chart of operations in another example method of synthesizing a nucleoside triphosphate analogue. Referring now to FIG. 13, a method for synthesizing a nucleoside triphosphate analogue may include reacting a protected nucleoside with a phosphitylating agent and pyrophosphate to form a first intermediate (operation 1310). In various examples, the protected nucleoside may include a sugar, a nucleobase, and a protected 3′-O group. The sugar may include any suitable sugar, for example such as described with reference to FIG. 1 or elsewhere herein. The nucleobase may include any suitable naturally occurring nucleobase, or any suitable non-naturally occurring nucleobase, such as described with reference to FIG. 1 or elsewhere herein. In some examples, the protected 3′-O group may include tert-butyldiphenylsilyl (TBDPS). The protected 3′-O group may inhibit the 3′ oxygen of the sugar from reacting with the phosphitylating agent or the pyrophosphate. In one nonlimiting example, the phosphitylating agent may include salicyl chlorophosphite.

With continued reference to FIG. 13, the second intermediate may then be reduced using a borane to form a boranophosphate intermediate (operation 1320). In various examples, the borane may include borane dimethylsulfide. With continued reference to FIG. 13, the boranophosphate intermediate may then be protected to form a protected boranophosphate intermediate (operation 1330). In various examples, the boranophosphate intermediate may be protected using bis(trimethylsilyl)acetamide (BSA). In various examples, the protected boranophosphate intermediate may include a trimethylsilyl group (TMS).

With continued reference to FIG. 13, the protected boranophosphate intermediate may then be activated and reacted with a reagent that includes a substituent, to form a second intermediate that includes a protected triphosphate group that includes the substituent (operation 1340). Nonlimiting reagents for activating and reacting the protected boranophosphate intermediate are described with reference to FIG. 8. The substituent may include any suitable fluorescent dye, alkyl chain, or polymer, for example such as discussed with reference to first substituent 118 in FIG. 1. In various examples such as described with reference to FIG. 8, the reagent may include an alcohol group which is coupled to the substituent (in which case the substituent may be coupled to the protected triphosphate group via an oxygen heteroatom, e.g., at the alpha phosphate), or an amine group which is coupled to the substituent (in which case the substituent may be coupled to the protected triphosphate group via a nitrogen heteroatom, e.g., at the alpha phosphate). With continued reference to FIG. 13, the second intermediate may then be deprotected to form a triphosphate group which includes the substituent (operation 1350).

### Additional Comments

It is to be understood that any respective features/examples of each of the aspects of the disclosure as described herein can be implemented together in any appropriate combination, and that any features/examples from any one or more of these aspects can be implemented together with any of the features of the other aspect(s) as described herein in any appropriate combination to achieve the benefits as described herein.

While various illustrative examples are described above, it will be apparent to one skilled in the art that various changes and modifications can be made therein without departing from the invention. The appended claims are intended to cover all such changes and modifications that fall within the true spirit and scope of the invention.

1. A nucleoside triphosphate analogue, comprising:
a sugar;
a nucleobase coupled to the sugar;
a triphosphate group coupled to the sugar;
a heteroatom coupled to an alpha phosphate of the triphosphate group; and
a first substituent coupled to the heteroatom.

2. The nucleoside triphosphate analogue of claim 1, wherein the heteroatom is selected from the group consisting of oxygen, nitrogen, and carbon.

3. The nucleoside triphosphate analogue of claim 1, wherein the first substituent comprises at least one of a fluorescent dye, an alkyl chain, and a polymer.

4. The nucleoside triphosphate analogue of claim 3, wherein the polymer comprises at least one of a synthetic peptide and a synthetic polymer.

5. The nucleoside triphosphate analogue of claim 4, wherein the synthetic polymer comprises an ethylene glycol polymer.

6. The nucleoside triphosphate analogue of claim 3, wherein the polymer comprises about 1 to about 50 repeating units.

7. The nucleoside triphosphate analogue of claim 1, wherein the first substituent comprises a functional group.

8. The nucleoside triphosphate analogue of claim 7, wherein the functional group comprises at least one of an amine, an azide, a carboxylic acid, a thiol, a tetrazine, a cyclooctyne, and an alkyne.

9. The nucleoside triphosphate analogue of claim 1, wherein the sugar comprises a natural ribose or deoxyribose.

10. The nucleoside triphosphate analogue of claim 1, wherein the nucleoside triphosphate analogue further comprises a second substituent coupled to the sugar via a 3′ carbon of the sugar.

11. The nucleoside triphosphate analogue of claim 1, wherein the second substituent comprises a protecting group.

12. The nucleoside triphosphate analogue of claim 11, wherein the protecting group comprises at least one of a vinyl group, an allyloxy methyl group, and an azido methyl group.

13. The nucleoside triphosphate analogue of claim 1, wherein the nucleoside triphosphate analogue comprises a third substituent coupled to the sugar via a 2′ carbon of the sugar.

14. The nucleoside triphosphate analogue of claim 13, wherein the third substituent comprises at least one of a fluorine, a methoxy group, and an ethoxy group.

15. The nucleoside triphosphate analogue of claim 1, wherein the nucleoside triphosphate analogue further comprises a fourth substituent coupled between a beta phosphate and a gamma phosphate of the triphosphate group.

16. The nucleoside triphosphate analogue of claim 15, wherein the fourth substituent comprises at least one of an oxygen atom, a nitrogen atom, a methylene, a fluoromethylene, a difluoromethylene, a sulfur atom, and a selenium atom.

17. The nucleoside triphosphate analogue of claim 1, wherein the nucleobase is a non-naturally occurring nucleobase.

18. A method of synthesizing a nucleoside triphosphate analogue, the method comprising:
coupling a phosphorylating agent to a sugar of a nucleoside, the nucleoside comprising the sugar and a nucleobase, the phosphorylating agent comprising a protecting group and a substituent;
oxidizing the coupled phosphorylating agent;
removing the protecting group from the oxidized, coupled phosphorylating agent to form a monophosphate diester comprising the substituent;
activating the monophosphate diester; and
reacting the activated monophosphate diester with a pyrophosphate to form a triphosphate group comprising the substituent.

19. The method of claim 18, wherein the sugar comprises a protected 3′-O group.

20-32. (canceled)

33. A method of synthesizing a nucleoside triphosphate analogue, the method comprising:
activating a monophosphate group of a nucleotide, the nucleotide comprising a sugar, a nucleobase, and the monophosphate group;
reacting the activated monophosphate group with an amidation agent comprising a substituent in an amidation reaction to form an amidated monophosphate group comprising the substituent;
activating the amidated monophosphate group; and
reacting the activated, amidated monophosphate group with a pyrophosphate to form a triphosphate group comprising the substituent.

34-85. (canceled)
